(19) United States
(12) Patent Application Publication
(10) Pub. No.: US 2004/0009962 A1
(43) Pub. Date: Jan. 15, 2004

(54) Combined estrogen blockade of the breast with exemestane and raloxifene

(76) Inventor: Maura N. Dickler, New York, NY (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/363,244

(22) PCT Filed: Sep. 4, 2001
(86) PCT No.: PCT/US01/27308

(30) Foreign Application Priority Data
Sep. 8, 2000 (US) 09657670

(51) Int. Cl. A61K 31/56; A61K 31/445

(52) U.S. Cl. 514/171; 514/319

(57) ABSTRACT

The invention provides methods and compositions for the treatment, prevention, and inhibition of breast cancer.

### TECHNICAL AREA OF THE INVENTION

This invention relates to methodologies for the treatment, prevention, and inhibition of breast cancer in mammals.

### BACKGROUND OF THE INVENTION

Breast cancer is the most common malignancy among women in the United States, and is second only to lung cancer as the most common cause of cancer related mortality. Landis et al. CA Cancer J. Clin. 48:6-29 (1998). It was estimated that 178,800 new cases of invasive breast cancer would be diagnosed in 1998, as well as an additional 36,900 new cases of ductal carcinoma in situ. Although mortality rates from breast cancer declined by approximately 1.8% per year between 1990 and 1994, approximately 43,500 women are still expected to die from breast cancer in 1999. Widespread screening and improvements in treatment, particularly with the use of adjuvant chemotherapy and hormonal therapy, have contributed to this declining mortality. However, new strategies for the treatment and prevention of breast cancer are needed if mortality is to continue to be significantly reduced in the future.

Tamoxifen (Nolvadex, ICI 46,474) is a nonsteroidal anti-estrogenic compound that was initially approved in the United States in 1977 for the treatment of postmenopausal women with advanced breast cancer. Jaiyesimi et al. J. Clin. Oncol. 13:513-529 (1995). Tamoxifen remains the first line of hormonal therapy for primary and recurrent breast cancer. Its biological effects are mediated through its binding to the estrogen receptor (ER), with subsequent inhibition of the actions of estrogen. The tamoxifen/ER-complex prevents estrogen-induced gene expression, leading to the inhibition of the phenotypic effects of estrogen on breast cancer cells.

In clinical trials, tamoxifen has been shown to induce objective response rates of 30 to 40% in unselected postmenopausal women with metastatic disease. Robert, Oncology, 11(S1):15-19 (1997). Response rates increase to 60-70% in women with ER-positive and progesterone receptor (PR)-positive tumors. Tamoxifen has also been shown to be effective for the treatment of metastatic breast cancer in premenopausal women, with response rates ranging between 20 to 45%. Ingle et al J. Clin. Oncol. 4:178-185 (1986); Buchanan et al. J. Clin. Oncol. 4:1326-1330 (1986). For the treatment of early stage breast cancer, tamoxifen has been shown to reduce the annual rates of recurrence and mortality when used as adjuvant therapy in both premenopausal and postmenopausal women. Early Breast Cancer Trialists' Collaborative Group, Lancet, 351:1451-1467 (1998). In addition, five years of adjuvant tamoxifen was demonstrated to reduce the risk of contralateral breast cancer by 47%, as compared with women who received no adjuvant therapy.

Although tamoxifen has been shown to reduce the risk of invasive and non-invasive breast cancer, tamoxifen is associated with an increased incidence of endometrial carcinoma. The risk of invasive endometrial cancer is increased about 2.5 fold among tamoxifen-treated women, with this risk concentrated in women greater than age 50.

In the Pilot Breast Cancer Prevention Trial at the Royal Marsden Hospital transvaginal ultrasound and endometrial biopsies were performed to evaluate the effects of tamoxifen compared to placebo on the postmenopausal endometrium and ovaries. Kedar et al. Lancet, 343:1318-1321 (1994); Powles et al. Oncology, 12(S5):28-31 (1998). Endometrial thickness was increased in 39% of the tamoxifen-treated group as compared with 10% in the placebo-treated group. In addition, the endometrial biopsies in the tamoxifen-treated women were remarkable for endometrial proliferation, a typical hyperplasia, and polyps. In this small pilot trial, no cases of endometrial carcinoma were observed, and no difference in the incidence of ovarian cysts was detected between the two groups.

Effective treatments and preventative therapies for breast cancer are needed that do not increase the risk of endometrial cancer.

### SUMMARY OF THE INVENTION

It is an object of the invention to provide methods and compositions useful in the treatment, inhibition, and prevention of breast cancer. This and other objects of the invention are provided by one or more of the embodiments described below.

In one broad aspect, the invention provides a method of treating, preventing, or inhibiting breast cancer comprising administering raloxifene and exemestane to a mammal in combination therapy.

In a preferred embodiment, the invention provides a pharmaceutical composition comprising about 2.4 parts by weight of raloxifene and about 1 part by weight of exemestane.

The invention therefore provides a safe and effective therapy for the treatment, prevention, or inhibition of breast cancer, without an increased risk of endometrial cancer.

### DETAILED DESCRIPTION OF THE INVENTION

Raloxifene

Raloxifene is one of a new generation of nonsteroidal anti-estrogens, referred to as selective estrogen receptor modulators (SERMs), with tissue specific estrogen agonist and antagonist effects. Gradishare et al. J. Clin. Oncol. 15:840-852 (1997). Similar to tamoxifen, raloxifene acts as an estrogen agonist in the bone and cardiovascular tissues, and as an estrogen antagonist in the breast. In contrast to tamoxifen, raloxifene acts as an estrogen antagonist in the endometrium, and can serve as a safer alternative to tamoxifen as a chemoprevention agent.

Raloxifene binds to the ligand-binding domain of an estrogen receptor, inducing an alternative conformational change with differential activation of distinct receptor domains. Brzozowski et al. Nature, 389:753-758 (1997). The raloxifene-ER complex may bind to an alternative DNA response element, the raloxifene response element (RRE), altering gene activation pathways. Yang et al. Science, 273:1222-1225 (1996). The resulting differential expression of estrogen-regulated genes may account for the tissue specific effects.

Raloxifene has been shown to have anti-tumor activity in the DMBA- and NMN-induced rat model of mammary carcinogenesis, although the anti-tumor activity may be less when compared with tamoxifen. Clemens et al. Life Sciences, 32:2869-2875 (1983); Gottardis & Jordan, Cancer Res. 47:4020-4024 (1987). Raloxifene inhibits the uterotropic action of estradiol in the immature rat uterine weight test, and has little agonist activity on the rat uterus when administered alone. Black et al. Life Sciences 32:1031-1036 (1983). In addition, raloxifene increases bone mineral density and decreases serum cholesterol in the ovariectomized rat. Black et al. J. Clint. Invest. 93:63-69 (1994). There have been no reports of DNA adducts or hepatocarcinogenesis in rats caused by raloxifene.

In a phase I trial of raloxifene, 200 mg/day was administered orally to healthy men. Draper et al. Pharmacology, 50:209-217 (1995). There was evidence of an anti-estrogen effect, as raloxifene was shown to blunt the response to exogenous estrogen. In a phase II trial, 14 patients with metastatic breast cancer who had previously received tamoxifen were treated with raloxifene at 200 mg/day. Buzdau, et al. Oncology, 45:344-345. The drug was well tolerated, with no significant clinical or laboratory abnormalities. However, no objective responses were observed in this tamoxifen-refractory group.

Raloxifene was further evaluated in a cohort of healthy postmenopausal women to determine its effects on bone mineral density, markers of bone turnover, serum cholesterol, and endometrial stimulation. Delmas et al. N. Engl. J. Med. 337:1641-1647 (1997). At an interim analysis, all doses of raloxifene were demonstrated to increase bone mineral density in the hip, spine, femur and total body, as well as to decrease markers of bone turnover as compared to placebo-treated patients. In addition, raloxifene significantly decreased serum total and LDL cholesterol, although HDL cholesterol remained unchanged. Endometrial thickness was unaffected by raloxifene in any of the treatment groups through the study, as monitored by transvaginal ultrasound. The drug was well tolerated, with side effects limited to increased hot flashes and leg cramps. An analysis of raloxifene-treated women across all placebo-controlled clinical trials showed an increased risk of venous thromboembolic events defined as deep vein thrombosis, pulmonary embolism, and retinal vein thrombosis. Based on these results, raloxifene was approved by the FDA for the prevention of osteoporosis at a dose of 60 mg/day.

In an 8-week trial of raloxifene to evaluate the short-term effects of the drug, endometrial biopsies were performed in 251 women at baseline and after 8 weeks of treatment. Draper et al. J. Bone Miner. Res. 11:835-842 (1996). Uterine biopsies of raloxifene-treated subjects showed no change in the endometrium during this short-term treatment, however biopsies in the estrogen-treated group showed significant endometrial stimulation.

In a large osteoporosis trial designed to assess for the risk of fracture (Multiple Outcomes of Raloxifene Evaluation Trial (“MORE”)), 7704 postmenopausal women were randomized to receive raloxifene 60 or 120 mg/day, or placebo. Cummings et al. Proc. Am. Soc. Clin. Oncol. 17:2A (1998). The raloxifene-treated patients had a relative risk of breast cancer of 0.42 [CI: 0.25, 0.73] compared with controls, representing a risk reduction of 58%. Jordan et al. Proc. Am. Soc. Clin. Oncol. 17:122A (1998). Patients who received raloxifene for >18 months derived the greatest benefits, with a RR of 0.23 [CI 0.10, 0.49]. The greatest reduction in breast cancers were observed for ER-positive and/or PR-positive tumors. The MORE trial also revealed a reduction in the risk of endometrial cancer in the raloxifene-treated group, with a relative risk of 0.38 (p=0.232). If two cases of endometrial cancer diagnosed within one month of randomization were excluded, the estimate of relative risk was 0.13 (p=0.045).

Exemestane

Exemestane (FCE 24304) is a Type I aromatase inhibitor that was synthesized in the laboratories of Pharmacia & Upjohn. It is structurally related to the natural substrate, androstenedione, and is recognized as a substrate by the aromatase cytochrome P-450 enzyme. Exemestane is processed through the normal catalytic mechanism of the aromatase enzyme to a transformed product, which binds covalently and irreversibly to the catalytic site of the enzyme, causing its inactivation. Exemestane acts as a “suicide” inhibitor, with irreversible inactivation of the aromatase enzyme due to the catalytic transformation of the drug. Resumption of estrogen production depends on the de novo synthesis of new aromatase enzyme molecules. This mechanism of action differs from the reversible non-steroidal aromatase inhibitors, anastrozole and letrozole (Femara®).

The effect of exemestane on in vivo aromatization was studied in 10 postmenopausal women with advanced breast cancer using radiolabeled [³H]androstenedione and [¹⁴C]estrone. Treatment with exemestane suppressed whole body aromatization from a mean pretreatment value of 2.059% to 0.042% (mean suppression 97.9%). Geisler et al. Clinical Cancer Res. 4:2089-2093 (1998). Exemestane is more effective than the first and second-generation aromatase inhibitors (e.g. aminoglutethimide (Di Salle et al., Preclinical and clinical pharmacology of the aromatase inhibitor exemestane (FCE 24304), In Motta & Serio (Eds.) Sex Hormones and Antihormones in Endocrine Dependent Pathology: Basic and Clinical Aspects. Elsevier Science B.V. 1994 p. 303-309), and has comparable activity to anastrozole and letrozole, two highly potent aromatase inhibitors belonging to the triazole, nonsteroidal class.

Exemestane is highly effective against 7, 12-dimethyl-benzanthracene (DMBA)-induced mammary tumors in rats. Zaccheo et al. Cancer Chemother. Pharmacol. 23:47-50 (1989). Animal toxicity studies have shown good tolerability, except at relatively high doses. Reproduction studies have been carried out to evaluate the effects of exemestane on fertility and reproductive performance in rats and embryotoxicity in rats and rabbits. All results indicate that exemestane is not teratogenic in these species. It is embryotoxic from 50 and 270 mg/kg in rats and rabbits, respectively, and can cause delivery complications.

Mutagenicity studies in vitro and in vivo have been conducted in order to evaluate the genotoxic potential of exemestane. Among the tests performed (Ames, V79, E. coli, DNA repair on rat hepatocytes, micronucleus test, and in vivo and in vitro chromosome aberration), all were negative except the chromosome aberration test in human lymphocytes. This test was positive starting from 12 μg/ml, i.e. a concentration much higher than that expected in human plasma following therapeutic doses.

A total of 8 phase I clinical pharmacology studies have been carried out with exemestane. The drug was administered in single doses to 41 postmenopausal volunteers in 2 trials and daily for 1 week to 32 postmenopausal volunteers in another study. One hundred and seventy-three postmenopausal patients with advanced breast cancer received chronic treatment with exemestane in 5 further studies: 48 patients received the drug weekly and 125 on a daily basis (intrapatient dose escalation in 30 and fixed daily dose in 95 patients). Zilembo et al., Proc. XVI International Cancer Congress, New Delhi, India, (1994); Howell et al. Eur. J. Cancer Clin. Oncol. 24:1567-1572 (1988); Robertson et al. Eur. J. Cancer. Clin. Oncol. 25:469-475 (1989); Dixon et al. Br. J. Cancer, 62:868-870 (1990). In general, inhibition of plasma estrogen levels was apparent starting at a dose of about 1 mg with suppression of E₂, E₁, and E₁S. Evans et al. Cancer Res. 52:5933-5939 (1992); Zilembo et al. Proc. XVI International Cancer Congress, New Delhi, India (1994); Bajetta et al. Eur. J. Cancer, 33:587-591 (1997).

Several Phase II trials of exemestane have been performed in postmenopausal women with metastatic breast cancer as second- and third-line hormonal therapy after treatment with tamoxifen. In one study, 25 mg of exemestane was administered to 128 postmenopausal women with tamoxifen-refractory disease. Jones et al., Abstract 436, 21^{st} Ann. San Antonio Breast Cancer Symposium, San Antonio, Tex. (1998). There was an overall response rate (CR+PR) of 28% (95% CI 21-37), which was increased to 34% in the 88 patients who had measurable disease. In addition, stable disease>6 months was seen in 19% of the treated population, producing an overall success rate (CR+PR+SD>6 months) of 47%. Median duration of response was 14 months (95% CI 11-20 months). Visceral disease was present in 52% of the treated cohort, and an overall response rate of 33% was seen in this group.

In another phase II trial, 87 postmenopausal women with metastatic breast cancer refractory to tamoxifen and megace were given exemestane 25 mg/day. Jones et al. Abstract 437, 21^{st} Ann. San Antonio Breast Cancer Symposium, San Antonio, Tex. (1998). The overall response rate was 11% (95% CI 6-20), with SD>6 months observed in an additional 17.2% of patients.

The antitumor efficacy of exemestane, 25 mg/day orally, was recently evaluated in 241 postmenopausal women with metastatic breast cancer whose tumors had failed prior non-steroidal aromatase inhibitors (aminoglutethimide in 56%, and other aromatase inhibitors including anastrozole, vorozole and letrozole in 44%). Lonning et al. Abstract 435, 21^{st} Ann. San Antonio Breast Cancer Symposium, San Antonio, Tex. (1998). All patients had received at least 2 prior hormonal regimens, and 23% had received 3 or more hormonal regimens. The overall objective response rate was 7%, with an overall success rate (CR+PR+SD>6 months) of 25%. These results are surprisingly favorable since exemestane was administered as third- and fourth-line therapy to women previously exposed to non-steroidal aromatase inhibitors. Exemestane may have the potential advantage of being a steroidal aromatase inhibitor, acting as an irreversible inhibitor of the enzyme.

Combination of Raloxifene and Exemestane

It is a discovery of the present invention that a combined estrogen blockade of the mammalian breast with raloxifene (an anti-estrogen) and exemestane (an aromatase inhibitor) can be more effective for treatment, prevention, and inhibition of breast cancer than administration of an anti-estrogen alone. Further, the combination of raloxifene and exemestane can be more effective than the use of tamoxifen and an aromatase inhibitor.

In postmenopausal women, the majority of circulating estrogen is synthesized from the peripheral conversion of androgens (androstenedione and testosterone) to estrogens (estrone and estradiol). The rate-limiting enzyme responsible for the conversion is aromatase P450 cytochrome. This process of aromatization occurs at peripheral sites including theadipose tissue, muscle, and liver. Harvey, Oncology, 12:32-35 (1998). Aromatase activity has also been detected in the breasts of women with both benign and malignant disease. O'Neill et al. Br. J Cancer, 56:601-604 (1987).

Breast tumor estrogens are increased in the majority of cancers, with breast tissue estrogen concentrations significantly higher in malignant breast tissue than in nonmalignant tissue. In addition, the concentrations of estrogens in breast tumor tissue in postmenopausal patients are much higher than expected, and are similar to those in premenopausal patients despite the lower circulating estrogens in the postmenopausal population. Van Landeghem et al. Cancer Res. 49:2900-2906 (1985). Therefore, a breast tumor tissue-plasma gradient exists in postmenopausal women, with an estimated tissue: plasma ratio of 10-50:1.

This greater-than-expected tissue estrogen concentration in postmenopausal breast cancer may be secondary either to increased uptake of estrogen from plasma or to in situ estrogen production within the tumor. Yue et al. demonstrated the importance of in situ aromatization versus uptake of peripheral estrogens as a mechanism for the high tumor estrogen concentration. Cancer Res. 58:927-932 (1998).

Anti-estrogens appear to be more effective in inhibiting ER-positive breast cancer cell lines in a low estrogen environment, and aromatase inhibitors can reduce circulating and breast tissue estrogens to enhance the low estrogen state. For example, in ovariectomized and intact athymic nude mice that were inoculated with MCF-7 breast cancer cells, growth of the tumor cells was dependent upon the presence of estrogen in a dose-dependent fashion. Osborne et al. Cancer Res. 45:584-590 (1985). In established tumors, initially grown in the presence of estrogen, the combination of tamoxifen and raloxifene only slowed the continued growth of these tumors. However, with estrogen deprivation, the combination of tamoxifen and raloxifene caused cessation of tumor growth, although no tumor regression was seen. There was a marked reduction in the tumor mitotic rate in tamoxifen-treated mice in an estrogen deficient state as compared with tamoxifen-treated mice supplemented with estrogen. In the absence of estrogen, the combination of tamoxifen and raloxifene caused initial stimulation of tumor growth, followed by a prolonged stationary phase. Both anti-estrogens were more effective for in vivo tumor growth inhibition in a low estrogen environment.

Tamoxifen and exemestane has been administered to a DMBA-induced rat mammary tumor model. Zaccheo et al. J. Steroid Biochem. Molec. Biol. 44:677-680 (1993). Exemestane was administered alone or in combination with tamoxifen. A higher objective response rate of 57% was observed with the combination, as compared with exemestane or tamoxifen alone, with response rates of 44% and 29% respectively. The appearance of new tumors was reduced by each treatment alone, however, the combination of agents was most effective in the prevention of new tumors.

The combination of tamoxifen and anastrozole (ARimidex®), an aromatase inhibitor, has been evaluated in postmenopausal women with early stage breast cancer. Dowsett et al. Breast Cancer Res. and Treat. 46:30 (1997). Women already receiving tamoxifen as adjuvant therapy were randomized to receive anastrozole or placebo for 28 days. Estradiol levels were significantly reduced in patients who received both anastrozole and tamoxifen compared with tamoxifen-treated patients alone (p<0.001). Serum concentrations of anastrozole and tamoxifen were unaffected by the combination, which was well tolerated with minimal toxicity.

The combination of raloxifene and exemestane is a more effective strategy for the treatment, prevention, and inhibition of breast cancer than an anti-estrogen alone or than tamoxifen combined with an aromatase inhibitor, and it is also more effective. This combination treatment could impact on thousands of women who are at high risk for breast cancer each year.

Methods of Treatment

The combination of raloxifene and exemestane can be used to treat or prevent cancer, or to inhibit or reverse the growth of a cancerous cell or tumor. Preferably, the cancer is breast cancer. Prevention of breast cancer comprises both the primary prevention of breast cancer in mammals that have not yet developed breast cancer and secondary prevention of breast cancer, i.e., the prevention of second primary tumors in mammals cured of an initial breast cancer, or the prevention of breast cancer in mammals who have had pre-malignant lesions. Preferably, the compositions and methods of the invention provide for secondary prevention of breast cancer.

The types of breast cancer that can be treated by the methods and compositions of the invention include invasive cancers (extending into the surrounding stroma) or non-invasive cancers (confined to ducts or lobules). Invasive breast cancers include, for example, infiltrating ductal carcinoma, infiltrating lobular carcinoma, infiltrating ductal and lobular carcinoma, medulary carcinoma, mucinous colloid carcinoma, comedocarcinoma, Paget's disease, papillary carcinoma, tubular carcinoma, and non-specific carcinomas and adenocarcinomas. Non-invasive carcinomas include, for example, intraductal carcinoma, lobular carcinoma in situ (LCIS), papillary carcinoma, and comedocarcinoma. The breast cancer to be treated can include estrogen receptor positive or estrogen receptor negative cancers and progesterone receptor positive or progesterone receptor negative cancers.

A combination of raloxifene and exemestane can be administered to a mammal, such as a mouse, rat, rabbit, guinea pig, macaque, baboon, chimpanzee, or human. The mammal may be a female or a male. Preferably, the mammal is a pre-menopausal or post-menopausal human female. Oral administration is contemplated, however, the raloxifene and exemestane can be delivered by any means known in the art, including intramuscular, intradermal, intraperitoneal, intravenous, or subcutaneous injection. Additional administration methods include intranasal and intravaginal administration.

The amount of each component administered is determined by the attending clinician taking into consideration the etiology and severity of the disease, the patient's condition and age, the potency of each component and other factors. Preferably, a large mammal, such as a human, is administered from about between 5 and 350 mg of raloxifene per day and from about between 5 to 600 mg of exemestane per day. Even more preferably, a large mammal is administered from about between 10 and 250 mg of raloxifene per day and from about between 10 to 500 mg of exemestane per day. Still even more preferably a large mammal is administered from about between 20 and 200 mg of raloxifene per day and from about between 15 to 300 mg of exemestane per day. Yet even more preferably, a large mammal is administered about 60 mg of raloxifene a day and about 25 mg of exemestane per day.

Raloxifene and exemestane can each be administered separately (i.e., sequentially) or when the modes of administration are the same, both of them may be administered in the same composition, yet in any case the preferred ratio of raloxifene to exemestane administered daily will be about will be between about 1:1 to 5:1, and most preferably will be about 2.4:1.

Compositions

Raloxifene and exemestane may be formulated with conventional pharmaceutical excipients, e.g., spray dried lactose and magnesium stearate, into tablets or capsules or other conventional dosage forms for oral administration. The raloxifene and exemestane are typically compounded (separately or together) in customary ways for oral administration, e.g., in capsules, tablets, as dragees or even in liquid form, e.g., suspensions or syrups. One or both of the active substances, optionally along with one or more additional active agents, can be worked into tablets or dragee cores by being mixed with solid, pulverulent carrier substances, such as sodium citrate, calcium carbonate or dicalcium phosphate, or binders such as polyvinylpyrrolidone, gelatin or cellulose derivatives, and possibly by adding also lubricants such as magnesium stearate, sodium lauryl sulfate, carnauba wax, or polyethylene glycols. Of course, taste-improving substances can be added in the case of oral-administration forms.

The therapeutically active compounds should be present in a concentration of about 0.5-90% by weight of the total mixture, i.e., in amounts that are sufficient for maintaining the above-mentioned range of dosage. Adjuvants can be added to any dosage form of the invention. Adjuvants include, but are not limited to, polyethylene glycol, polyvinylpyrrolidone, a medium chain triglyceride, a long chain triglyceride, and tocopherol acetate.

As farther forms of raloxifene and exemestane administration (separately or in combination), one can use plug capsules, e.g., of hard gelatin, as well as closed soft-gelatin capsules comprising a softener or plasticizer, e.g., glycerine. The plug capsules contain the active substance or substances preferably in the form of granulate, e.g., in mixture with fillers, such as lactose, saccharose, mannitol, starches, such as potato starch or amylopectin, cellulose derivatives or highly-dispersed silicic acids. In soft-gelatin capsules, the active substance is preferably dissolved or suspended in suitable liquids, such as vegetable oils or liquid polyethylene glycols. In place of oral administration, the active compounds may be administered (separately or in combination) parenterally. In such cases, a solution of the active substance, e.g., in sesame oil or olive oil can be used.

Methods of intranasal administration of raloxifene and exemestane (separately or in combination) in the form of nose drops or nasal spray are also contemplated by the invention. Formulations suitable for intranasal administration can consist of (a) liquid solutions, such as an effective amount of an active ingredient dissolved in diluents, such as water, or saline; (b) suspensions in an appropriate liquid; and (c) suitable emulsions, all of which can be administered in suitable ways, including nose drops and nasal sprays. Formulations can also include gels, ointments and the like, containing, in addition to the active ingredient, such excipients as are known in the art, all of which can be administered in suitable ways, including by painting on the nasal mucosa, or squirting into the nose. Raloxifene and exemestane can also be delivered (separately or in combination) via an intra-vaginal suppository. Typical carriers used in standardized suppositories are solid and meltable at human or animal body temperature. Examples of carriers include, but are not limited to, beeswax or glycerol or both.

The following are provided for exemplification purposes only and are not intended to limit the scope of the invention described in broad terms above. All references cited in this disclosure are incorporated herein by reference.

### EXAMPLE 1

Administration of Raloxifene and Exemestane to Human Breast Cancer Patients

Postmenopausal women with a history of estrogen receptor (ER)-negative and progesterone receptor (PR)-negative American Joint Committee on Cancer (AJCC) Stage I, II, or III invasive breast cancer, with no clinical evidence of the disease and completed adjuvant therapy are administered a combination of raloxifene and exemestane. The patients were randomized to either raloxifene at 60 mg orally each day (Group A) or exemestane at 25 mg orally each day (Group B) for 2 weeks (time designated: week-2). After 2 weeks of single agent therapy, patients were started on combination therapy with oral raloxifene (60 mg/day) and oral exemestane (25 mg/day) (time designated: week 0), and continued on both drugs for one year (month 12). Patients were required to start supplemental calcium (900-1500 mg/day) plus vitamin D (400-600 units/day) orally each day during week 0. Both raloxifene and exemestane could be administered without regard to meals.

For Group A, a plasma raloxifene level and plasma concentration of estradiol E₂), estrone (E₁), and estrone sulfate (E₁S) (referred to as “plasma estrogens”) was performed at baseline (week-2). After 2 weeks of single agent raloxifene (week 0), raloxifene level and plasma estrogen concentrations were drawn pre-dose, and then raloxifene levels were drawn at 2, 4, 6, and 8 hours after administration of raloxifene. Patients were instructed to start exemestane the next morning in combination with raloxifene. After 2 weeks of combination therapy, exemestane and raloxifene levels, as well as plasma concentration of estrogens, were drawn pre-dose. After administration of raloxifene and exemestane, levels of each drug were drawn at hours 2, 4, 6, and 8. The schedule of blood sampling starting at week 0 is summarized in Table 1.

TABLE 1

| | Week 0 (Single Agent Therapy) | | | | | Week 2 (Combination Therapy) | | | | |
|---|---|---|---|---|---|---|---|---|---|---|
| | Pre-Dose | 2 h | 4 h | 6 h | 8 h | Pre-Dose | 2 h | 4 h | 6 h | 8 h |
| Drug | X | X | X | X | X | X | X | X | X | X |
| Estrogens | X | | | | | X | | | | |

For Group B, a plasma exemestane level and plasma concentration of estradiol (E₂), estrone (E₁), and estrone sulfate (E₁S) were performed at baseline (week-2). After 2 weeks of single agent exemestane (week 0), an exemestane level and plasma estrogen concentrations was drawn pre-dose, and then an exemestane level was drawn at 2, 4, 6, and 8 hours after administration of exemestane. Patients were instructed to start raloxifene the next morning in combination with exemestane. After 2 weeks of combination therapy, exemestane and raloxifene levels, as well as plasma concentration of estrogens were drawn pre-dose. After administration of raloxifene and exemestane, levels of each drug were drawn at hours 2, 4, 6, and 8. The schedule of blood sampling is summarized in Table 1.

At months 3, 6, and 12, exemestane and raloxifene levels and plasma concentrations of estrogens were obtained prior to the daily dose to ensure compliance and suppression of estrogens.

### EXAMPLE 2

Parameters Measured During Treatment

Measurement of Plasma Estrogens

Patients were instructed not to take the assigned drug(s) on the days of sampling until the blood was sampled. Blood was collected in precooled Li-Heparin tubes in order to avoid exemestane degradation. After blood collection, the tubes were placed at 4° C. Samples were centrifuged within 30 minutes from collection at 1200×g for 10 min. at 4° C.

Plasma estrogens were measured by HPLC/RIA using the procedure described by Johannessen et al. Clin. Cancer Res. 3:1101-1108 (1997). Briefly, a 2 ml plasma sample was loaded onto a preconditioned Amprep C18 cartridge, then serially washed with 4 ml of 24% acetonitrile in water, to collect E₁S, and 4 ml of 100% acetonitrile, to collect E₁ and E₂. E₁S was then deconjugated to E₁ with arylsulfatase. The samples were then purified by HPLC, using a C18 column. The fractions containing individual E₁ and E₂, or deconjugated E₁, were collected, evaporated, and stored at −20° C. until performance of a specific RIA.

Exemestane and Raloxifene Plasma Levels

Exemestane was assayed in plasma using a validated liquid chromatographic method with tandem mass spectrometry detection. Briefly, the extraction of the compound was performed by solid phase extraction. A Zorbax SB C8 column (4.6×150 mm, 5 μm), or equivalent, was used to perform the chromatographic separation using acetonitrile as the mobile phase. MS detection was realized using the Heated Nebulizer interface, with multiple reaction monitoring (297→121 m/z for exemestane) operated in positive ion mode. The lower limit of quantification would be about 0.050-0.1 ng/ml.

A raloxifene assay was performed using a validated HPLC method characterized by the appropriate selectivity and limit of quantitation.

Markers of Bone Density

Bone mineral density (BMD) of the lumbar spine and total hip was measured by dual-energy X-ray absorptiometry at baseline and after 12 months of combination therapy. Whenever possible, each patient would have a follow-up BMD on the same scanner. Sites included the average lumbar spine (L1-L4) and the femoral neck.

A spot urine for N-telopeptide, calcium, and creatinine was performed at baseline, and every 3 months for the duration of therapy (months 3, 6, 9, and 12). These are measures of osteoclastic activity. Serum bone specific alkaline phosphatase (a measure of osteoblastic activity) was drawn at baseline and months 3, 6, 9, and 12.

Serum Lipids

Fasting blood for total cholesterol, HDL, LDL, and triglycerides were preformed at baseline, and months 6 and 12.

Quality of Life

Quality of life was measured by a modified version of the questionnaire used in the NSABP Breast Cancer Prevention Trial-P1. See Fisher et al., JNCI, 90:1371-1388 (1998). The questions assess symptoms of estrogen deficiency and sexual functioning, including the occurrence of hot flashes, vaginal discharge, vaginal dryness, fluid retention, nausea, skin changes, diarrhea, and weight gain or loss (43 questions in total). If patients experienced these symptoms during the past 3 months, they must indicate the severity of these symptoms on a scale of 1 to 4 (1=slightly, 4=extremely). This questionnaire was administered at baseline and at months 3, 6, and 12. Other symptoms relating to drug toxicity were captured at each 3 month visit during the history and physical exam, and were scored using the NCI Common Toxicity Scale.

Correlative Laboratory Studies

As an elective option, patients could have correlative laboratory studies performed on biopsy material obtained for the unaffected breast pre- and post-treatment (month 3). This was a core needle biopsy or small circumareolar incision with a biopsy of underlying breast tissue. Samples were processed for routine histo-pathological evaluation, and breast tissue aromatase activity and tissue estrogen levels. Optionally, immunohistochemical staining was performed including, ER, PR, Ki-67, her2/neu, EGFR, p53, DNA ploidy, and the tunnel assay for apoptosis.

During biopsy, a minimum of 1 g of normal breast tissue is taken and immediately frozen at −80° C. Breast tissue estrogens were measured similar to the method described by Van Landeghem et al. Cancer Res. 45:2900-2906 (1985). Briefly, an approximately 0.5 gram aliquot of tissue was pulverized at −196° C. with a microdismembrator. The powder was suspended in buffer and extracted with ethanol:acetone (1:1). The extract was evaporated, resuspended in 70% methanol in water and left overnight at −20° C. to allow separation of the lipids. Following centrifugation, the organic layer was evaporated, and redissolved in 1 ml of 2M acetic acid. The samples were then subjected to solid phase extraction and PLC purification for plasma estrogens.

Aromatase activity was assessed in breast tissue microsomes using the procedure described by de Jong et al. (Cancer Res. 57:2109-2111 (1997)) and Miller (J. Steroid Biochem. Molec. Biol. 39:783-790 (1991)), with minor modifications. Briefly, an approximately 0.5 gram aliquot of breast tissue was pulverized at −196° C. with a microdismembrator. The powder was suspended with phosphate buffer and centrifuged at 9,000×g. The supernatant was further ultracentrifuged at 105,000×g, and the pellet was suspended in buffer. An aliquot of the microsomal suspension was incubated with [1β-3H] androstenedione and cofactors for 2 hours at 37° C. The aromatase activity was determined by the formation of tritiated water released during the aromatization of the substrate.

CBC/Chemistries

Laboratory tests including CBC with differential, chemistry profile including BUN, creatinine, sodium, potassium, chloride, calcium, glucose, total bilirubin, total protein, albumin, alkaline phosphatase, and AST (SGOT) were performed at baseline, and at months 3, 6, 9, and 12. The bone specific alkaline phosphatase (mentioned in markers of bone turnover) would be included in this chemistry blood draw listed in Table 2.

Breast Imaging

Mammography and breast NM was performed at baseline and month 12. For women who elected to participate in the optional breast biopsy at month 3, a breast MRI was performed at that time to correlate breast tissue estrogen levels with quantitative changes in breast density by MRI imaging. Whenever possible, the breast MRI was performed prior to the breast biopsy.

All patients were followed for at least 12 months. See Table 2.

TABLE 2
Evaluation During Treatment

| | | Week | | | Month | Month | Month | Month |
|---|---|---|---|---|---|---|---|---|
| Test Type | Pretreatment | -2 | Week 0 | Week 2 | 3 | 6 | 9 | 12 |
| History/Physical | X | | | | X | X | X | X |
| Drug Levels | | X | X | X | X | X | | X |
| Plasma Estrogens | | X | X | X | X | X | | X |
| BMD | X | | | | | | | X |
| Urine Markers | X | | | | X | X | X | X |
| Lipids | X | | | | | X | | X |
| QOL | X | | | | X | X | | X |
| Breast Biopsy | X | | | | X | | | |
| CBC/Chem/ | X | | | | X | X | X | X |
| Bone AlkP | | | | | | | | |
| Mammo/MRI | X | | | | | | | X |

TABLE 3
Evaluation of Patients Who Continue Treatment for More than 1 Year

| | Month | Month | Month | Month | Month | Month | Month | Month |
|---|---|---|---|---|---|---|---|---|
| Test Type | 18 | 24 | 30 | 36 | 42 | 48 | 54 | 60 |
| History/Physical | X | X | X | X | X | X | X | X |
| BMD | | X | | X | | X | | X |
| Urine Markers | X | X | X | X | X | X | X | X |
| Lipids | | X | | X | | X | | X |
| QOL | | X | | X | | X | | X |
| CBC/Chem/ | X | X | X | X | X | X | X | X |
| Bone AlkP | | | | | | | | |
| Mammo/MRI | | X | | X | | X | | X |

We claim:

1. A method of treating, preventing, or inhibiting breast cancer comprising administering raloxifene and exemestane in combination to a mammal.

2. The method of claim 1 wherein the raloxifene and exemestane are administered daily.

3. The method of claim 2 wherein about 5 to 350 mg/day of raloxifene and about 5 to 600 mg of exemestane is administered.

4. The method of claim 2 wherein about 60 mg/day of raloxifene and about 25 mg/day of exemestane is administered.

5. The method of claim 1 wherein the mammal has breast cancer.

6. The method of claim 1 wherein the mammal does not have breast cancer.

7. The method of claim 1 wherein the breast cancer is selected from the group consisting of estrogen receptor positive, estrogen receptor negative, progesterone receptor positive, and progesterone receptor negative.

8. The method of claim 1 wherein the mammal is a human.

9. The method of claim 7, wherein in the human is selected from the group consisting of pre-menopausal women, post-menopausal women, and men.

10. The method of claim 1 wherein the raloxifene and exemestane are administered at the same time.

11. The method of claim 1 wherein the raloxifene and exemestane are administered sequentially.

12. The method of claim 1 wherein the raloxifene and exemestane are administered orally.

13. A pharmaceutical composition comprising about between 5 and 350 mg of raloxifene and from about between 5 to 600 mg of exemestane.

14. The pharmaceutical composition of claim 13, further comprising an adjuvant selected from the group consisting of polyethylene glycol, polyvinylpyrrolidone, a medium chain triglyceride, a long chain triglyceride, and tocopherol acetate.

15. The pharmaceutical composition of claim 13, wherein the composition is administered as a dosage form selected from the group consisting of a tablet, capsule, suspension, syrup, injectable solution, intranasal formulation, and suppository.

16. A pharmaceutical composition comprising about between 10 and 250 mg of raloxifene and from about between 10 to 500 mg of exemestane.

17. A pharmaceutical composition comprising about between 20 and 200 mg of raloxifene and from about between 15 to 300 mg of exemestane.

18. A pharmaceutical composition comprising about 60 mg of raloxifene and about 25 mg of exemestane.

19. A pharmaceutical composition comprising about 2.4 parts by weight of raloxifene and about 1 part by weight of exemestane.
